(19) United States
(12) Patent Application Publication
(10) Pub. No.: US 2024/0398890 A1
(43) Pub. Date: Dec. 5, 2024

(54) Method for Treatment of Microbial Infections

(71) Applicant: Government of the United States as represented by the Secretary of the Air Force, Wright-Patterson AFB, OH (US)

(72) Inventor: Oscar N. Ruiz, Bellbrook, OH (US)

(21) Appl. No.: 18/638,772

(22) Filed: Apr. 18, 2024

Related U.S. Application Data
(60) Provisional application No. 63/469,862, filed on May 31, 2023.

(51) Int. Cl. A61K 38/05; A61F 13/00; A61K 9/06; A61K 47/06

(52) U.S. Cl. CPC A61K 38/05; A61F 13/00063; A61K 9/06; A61K 47/06; A61F 2013/00676; A61F 2013/00719; A61F 2013/00906; A61F 2013/0091

(57) ABSTRACT

A method of treating microbial infections that entails producing a space over the infected site by covering such site with an impermeable material and then filling such space with an antimicrobial material (in the form of a solution, cream, gel, colloid, etc) comprising an efflux pump inhibitor soluble in a non-polar solvent is provided. The antimicrobial material can also be applied in multiple other ways such as a cream or released from a band-aid or gauze. Such treatment method may also be used with newly discovered antibiotics.

### CROSS-REFERENCE TO RELATED APPLICATIONS

The present application claims priority to U.S. Provisional Application Ser. No. 63/469,862 filed May 31, 2023, the contents of such provisional application hereby being incorporated by reference in its entry.

### RIGHTS OF THE GOVERNMENT

The invention described herein may be manufactured and used by or for the Government of the United States for all governmental purposes without the payment of any royalty.

### FIELD OF THE INVENTION

The present invention relates to methods of treating microbial infections.

### BACKGROUND OF THE INVENTION

Currently, microbial infections are treated with antibiotics but these are becoming increasingly ineffective as microorganisms are developing resistant to multiple antibiotics. Without effective antibiotic treatment, an infection can rapidly progress and become systemic leading to death. Unfortunately, the discovery of new antibiotics has basically stop as most antibiotic pathways are now known.

Applicants recognized that the solution to the problem might not lie in the discovery of new antibiotics but in new methods of treatment using current antibiotics. Applicants' recognition led to the discovery that microorganisms, including bacteria and fungi, can be susceptible to mildly toxic non-polar solvents such as hydrocarbons, but they have developed specific systems known as efflux pumps (e.g., RND efflux pumps, MFS pumps, and ABC pumps) to remove (extrude) the non-polar solvents out of the cell in order to maintain an internal cellular concentration that is not toxic. By developing an antimicrobial material that contains an efflux pump inhibitor targeting the microbial efflux pumps and an otherwise low toxicity non-polar solvent, the microbial cell becomes susceptible to the non-polar solvent by the action of the efflux pump inhibitor which blocks or compete the pumps effectively blocking the efflux of non-polar solvents which leads to the accumulation of non-polar solvent inside the microbial cells to toxic levels that lead to microbial growth inhibitor and microbial cell death. Thus, Applicants disclose a new way to treat infections that entails producing a space over the infected site by covering such site with an impermeable material and then filling such space with an antimicrobial material (in the form of a solution, cream, gel, colloid, etc) comprising an efflux pump inhibitor soluble in a non-polar solvent. The antimicrobial material can also be applied in multiple other ways such as a cream or released from a band-aid or gauze. Such treatment method may also be used with newly discovered antibiotics.

### SUMMARY OF THE INVENTION

The present invention relates to a method of treating microbial infections that entails producing a space over the infected site by covering such site with an impermeable material and then filling such space with an antimicrobial material (in the form of a solution, cream, gel, colloid, etc) comprising an efflux pump inhibitor soluble in a non-polar solvent. The antimicrobial material can also be applied in multiple other ways such as a cream or released from a band-aid or gauze. Such treatment method may also be used with newly discovered antibiotics.

### BRIEF DESCRIPTION OF THE DRAWINGS

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 depicts a top view of an embodiment of the method of use having an adhesive cover with a fillable space over the affected allowing the incorporation of antimicrobial solution or gel. (1): Affected Tissue; (2): Adhesive impervious material cover with empty space over infected area to be filled with antimicrobial material solution; (3): microbial cells in infected tissue area; (4): Non-polar solvent in antimicrobial material; (5): Efflux pump inhibitor in antimicrobial material.

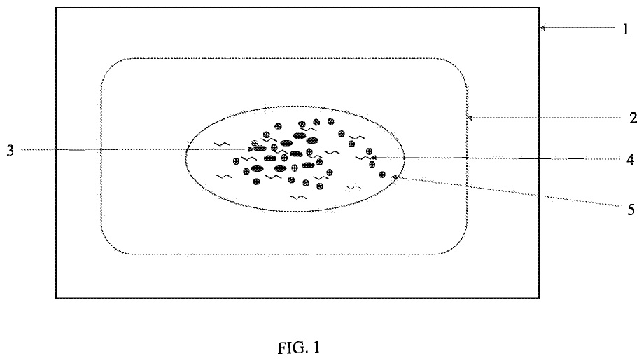

FIG. 2 depicts an embodiment of a method of use for application of antimicrobial in liquid solution over an affected area.

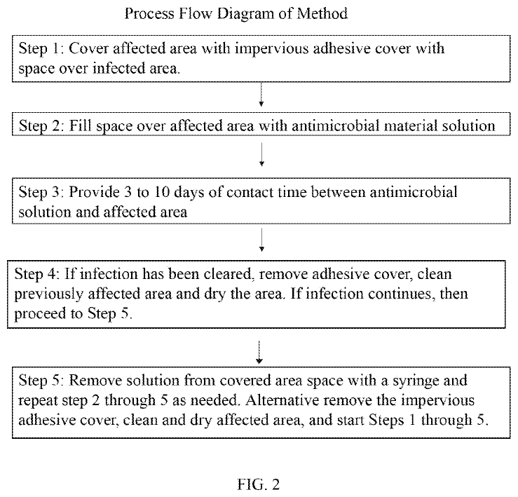

FIG. 3 depicts an embodiment of a method of use for application of antimicrobial in gel form over an affected area.

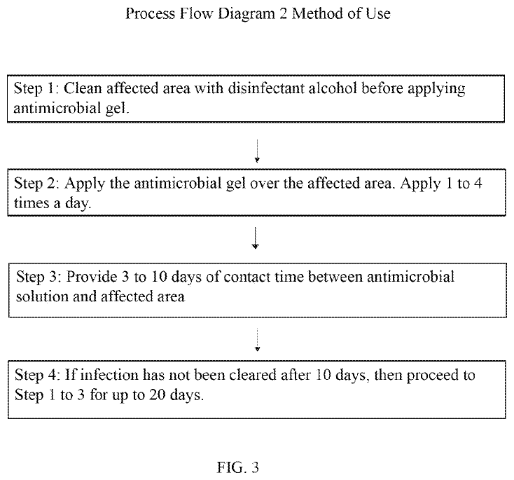

### DETAILED DESCRIPTION OF THE INVENTION

### Definitions

Unless specifically stated otherwise, as used herein, the terms “a”, “an” and “the” mean “at least one”.

As used herein, the terms “include”, “includes” and “including” are meant to be non-limiting.

As used herein, the words “about,” “approximately,” or the like, when accompanying a numerical value, are to be construed as indicating a deviation as would be appreciated by one of ordinary skill in the art to operate satisfactorily for an intended purpose.

As used herein, the words “and/or” means, when referring to embodiments (for example an embodiment having elements A and/or B) that the embodiment may have element A alone, element B alone, or elements A and B taken together.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Method Of Treating Microbial Infections

For purposes of this specification, headings are not considered paragraphs and thus this paragraph. In this paragraph, Applicants disclose a method comprising applying: applying a biocide to a target area and then applying a nonpolar solvent to said target area; applying a nonpolar solvent to a target area and then applying a biocide to said target area; or applying a mixture comprising a biocide and a nonpolar solvent to a target area; said biocide being a microbial efflux pump inhibitor. It should be noted that the application of the biocide and nonpolar solvent can not only reduce or eliminate an infection but can also prevent an infection.

Applicants disclose the method of the previous paragraph wherein said target area is an exterior or interior surface of a mammal. In one aspect, said target area is the skin, eye and/or ear of said mammal. In one aspect, said mammal is a human.

Applicants disclose the method of the previous two paragraph wherein said nonpolar solvent being selected from the group consisting of hydrocarbons and mixtures thereof; preferably said nonpolar solvent is selected from the group consisting of alkanes and mixtures thereof, more preferably said nonpolar solvent being selected from the group consisting of normal alkanes and mixtures thereof, most preferably said nonpolar solvent being selected from the group consisting of _medium chain n-alkanes and mixtures thereof.

Applicants disclose the method of the previous paragraph wherein said microbial efflux pump inhibitor is selected from the group consisting of: a compound configured to competitively bind to a biding site of the efflux pump, wherein the efflux pump is of the resistance nodulation division family; a compound configured to competitively bind to a binding site of the efflux pump, wherein the efflux pump is of the major facilitator superfamily; a compound configured to competitively bind to a binding site of the efflux pump, wherein the efflux pump is of the ATP-binding cassette superfamily; a molecule having a structure configured to recognize, interact, and block the efflux pump, preferably said molecule having a structure configured to recognize, interact, and block the efflux pump is selected from the group consisting of a pyridopyrimidine, an arylpiperazine, an arylpiperidine, peptidomimetic and mixtures thereof, preferably said peptidomimetic is a c-capped dipeptide, preferably a dipeptide compound, more preferably Phe-Arg-β-napthylamide and analogs thereof or a diamine-containing peptide and analogs thereof, a molecule that is configured to bind and block efflux pumps or porins within the cellular membranes, preferably said molecule that is configured to bind and block efflux pumps or porins within the cellular membranes is selected from the group consisting of biorecognition elements, antibodies, nanobodies, aptamers and mixtures thereof, preferably said aptamer is a nucleic acid; and a molecule that binds allostericly to an efflux pump, preferably said molecule that binds allostericly to an efflux pump is an allosteric inhibitor.

Applicants disclose the method of the previous four paragraphs wherein said wound is covered before said biocide and polar solvent are applied to said wound and wherein said biocide and polar solvent are applied under said covering.

Applicants disclose the method of the previous five paragraphs wherein said biocide and polar solvent are contained in a covering and said covering is applied to said wound.

Applicants disclose the method of the previous two paragraphs wherein said covering comprises a material selected from the group consisting of films, tapes, foams and woven or non-woven fabrics coated with acrylate, silicone or synthetic rubber and mixtures thereof, preferably said material is selected from the group consisting of medical single-coated tapes with liner, adhesive and carrier and double coated tapes with liner, adhesive, carrier and adhesive and mixtures thereof, more preferably said material is selected from the group consisting of medical hydrogels and hydrocolloids adhesives and mixtures thereof, most preferably said material is selected from the group consisting of medical grade silicone adhesive or medical grade silicon gel and mixtures thereof.

Applicants disclose the method of the previous seven paragraphs wherein said biocide and/or said nonpolar solvent are in the form of a cream or gel, preferably said each cream or gel has, independently, a viscosity of from about 0.5 cP to about 250,000 cP, more preferably said each cream or gel has, independently, a viscosity of from about 2,000 cP to about 50,000 cP, most preferably said each cream has, independently, a viscosity of from about 9,000 cP to about 16,000 cP.

Applicants disclose the method of the previous eight paragraphs wherein said wound is infected.

Applicants disclose the method of the previous nine paragraphs wherein said wound is infected with bacteria or fungi; preferably said bacteria is a Gram-negative bacteria or Gram-positive bacteria, and fungi is a filamentous fungus or yeast; more preferably said bacteria is a Pseudomonad or Acinetobacter, and fungus is a Candida group yeast; most preferably said bacteria is Pseudomonas aeruginosa, Acinetobacter baumannii, and fungus is Candida albicans.

Applicants disclose the method of the previous two paragraphs wherein said infection is reduced by at least 50%, preferably said infection is reduced by at least 75%, more preferably said infection is reduced by at least 85%, more preferably said infection is reduced by at least 95%, more preferably said infection is reduced by at least 99%, most preferably said infection is reduced by at least 99.99%.

Applicants disclose the method of the previous two paragraphs wherein said infection is prevented. In one aspect, said prevention sterilizes a surgical site before, during and/or after surgery.

### Test Methods

The effectiveness of the method for treatment of microbial infection is characterized via Microbial Enumeration Test (MET) following either ASTM D6974-20: Standard Practice for Enumeration of Viable Bacteria and Fungi in Liquid Fuels—Filtration and Culture procedure, or USP-NF M98800_01_01: Microbiological Examination of Nonsterile Products: Microbial Enumeration Test.

The viscosity of antimicrobial creams and antimicrobial gels are measured following the ASTM D445-21e2: Standard Test Method for Kinematic Viscosity of Transparent and Opaque Liquids (and Calculation of Dynamic Viscosity).

The antimicrobial treatment should provide a reduction of at least 50% from the initial infection level for disease treatment applications.

The antimicrobial treatment when used as a biostatic treatment for infection prevention will prevent infection in at least 50% of individuals.

### EXAMPLES

The following examples illustrate particular properties and advantages of some of the embodiments of the present invention. Furthermore, these are examples of reduction to practice of the present invention and confirmation that the principles described in the present invention are therefore valid but should not be construed as in any way limiting the scope of the invention.

Example 1: An affected area infected with bacteria, fungi or both is covered with an impervious adhesive cover (e.g., band aid) providing an empty space over the infected area. The empty space is filled with the antimicrobial solution, comprised of a combination of efflux pump inhibitor and non-polar solvent (e.g., dodecane), using a sterile syringe, pipette, or other devices. The antimicrobial solution is allowed to contact the affected area for approximately 3 to 10 days. After 3 to 10 days, the affected area is tested via microbial enumeration test (MET) to determine if the infection has been eliminated or reduced at least 90% from the initial level. If 90% reduction of infection has not been achieved, the adhesive cover is removed, the affected area cleaned with 70% isopropyl alcohol, and the procedure repeated as previously described. After approximately 3 to 10 days contact time the infection level is measured via MET and the process repeated as needed.

Example 2: An antimicrobial gel or antimicrobial cream comprised of efflux pump inhibitor and non-polar solvent (e.g., dodecane) is delivered to an affected area infected with bacteria, fungi or both approximately between 1 to 4 times per day area. The antimicrobial gel or cream solution are used for approximately 3 to 10 days. After 3 to 10 days, the affected area is tested via microbial enumeration test (MET) to determine if the infection has been eliminated or reduced at least 90% from the initial level. If 90% reduction of infection has not been achieved, the affected area is cleaned with 70% isopropyl alcohol, and the procedure repeated as previously described. After approximately 3 to 10 days contact time the infection level is measured via MET and the process repeated as needed.

Example 3: An antimicrobial liquid drop comprised of efflux pump inhibitor and non-polar solvent (e.g., dodecane) is delivered to an affected area infected with bacteria, fungi or both approximately between 1 to 4 times per day area. The antimicrobial drop is used for approximately 3 to 10 days. After 3 to 10 days, the affected area is tested via microbial enumeration test (MET) to determine if the infection has been eliminated or reduced at least 90% from the initial level. If 90% reduction of infection has not been achieved, the affected area is cleaned with 70% isopropyl alcohol, and the procedure repeated as previously described. After approximately 3 to 10 days contact time the infection level is measured via MET and the process repeated as needed.

Every document cited herein, including any cross-referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While the present invention has been illustrated by a description of one or more embodiments thereof and while these embodiments have been described in considerable detail, they are not intended to restrict or in any way limit the scope of the appended claims to such detail. Additional advantages and modifications will readily appear to those skilled in the art. The invention in its broader aspects is therefore not limited to the specific details, representative apparatus and process, and illustrative examples shown and described. Accordingly, departures may be made from such details without departing from the scope of the general inventive concept.

What is claimed is:

1. A method comprising applying:
a) applying a biocide to a target area and then applying a nonpolar solvent to said target area;
b) applying a nonpolar solvent to a target area and then applying a biocide to said target area; or
c) applying a mixture comprising a biocide and a nonpolar solvent to a target area said biocide being a microbial efflux pump inhibitor.

2. The method of claim 1 wherein said target area is an exterior or interior surface of a mammal.

3. The method of claim 2 wherein said target area is the skin, eye and/or ear of said mammal.

4. The method of claim 2 wherein said mammal is a human.

5. The method of claim 1 wherein said nonpolar solvent comprises one or more hydrocarbons.

6. The method of claim 5 wherein said nonpolar solvent comprise a normal alkane.

7. The method of claim 1 wherein said microbial efflux pump inhibitor is selected from the group consisting of:
a) a compound configured to competitively bind to a biding site of the efflux pump, wherein the efflux pump is of the resistance nodulation division family;
b) a compound configured to competitively bind to a binding site of the efflux pump, wherein the efflux pump is of the major facilitator superfamily;
c) a compound configured to competitively bind to a binding site of the efflux pump, wherein the efflux pump is of the ATP-binding cassette superfamily;
d) a molecule having a structure configured to recognize, interact, and block the efflux pump;
e) a molecule that is configured to bind and block efflux pumps or porins within the cellular membranes; and
f) a molecule that binds allostericly to an efflux pump, preferably said molecule that binds allostericly to an efflux pump is an allosteric inhibitor.

8. The method of claim 7 wherein said molecule having a structure configured to recognize, interact, and block the efflux pump is selected from the group consisting of a pyridopyrimidine, an arylpiperazine, an arylpiperidine, peptidomimetic and mixtures thereof, and said molecule that is configured to bind and block efflux pumps or porins within the cellular membranes is selected from the group consisting of biorecognition elements, antibodies, nanobodies, aptamers and mixtures thereof.

9. The method of claim 8 wherein said peptidomimetic is a c-capped dipeptide and said aptamer is a nucleic acid.

10. The method of claim 9 wherein said c-capped dipeptide is a dipeptide compound.

11. The method of claim 10 wherein said dipeptide compound is Phe-Arg-β-napthylamide and analogs thereof or a diamine-containing peptide and analogs thereof.

12. The method of claim 1 wherein said wound is covered before said biocide and polar solvent are applied to said wound and wherein said biocide and polar solvent are applied under said covering.

13. The method of claim 1 wherein said biocide and polar solvent are contained in a covering and said covering is applied to said wound.

14. The method of claim 12 wherein said covering comprises a material selected from the group consisting of films, tapes, foams and woven or non-woven fabrics coated with acrylate, silicone or synthetic rubber and mixtures thereof.

15. The method of claim 14 wherein said material is selected from the group consisting of medical hydrogels and hydrocolloids adhesives and mixtures thereof.

16. The method of claim 14 wherein said material is selected from the group consisting of medical grade silicone adhesive or medical grade silicon gel and mixtures thereof.

17. The method of claim 1 wherein said biocide and/or said nonpolar solvent are in the form of a cream or gel, preferably said each cream or gel has, independently, a viscosity of from about 0.5 cP to about 250,000 cP.

18. The method of claim 1 wherein said wound is infected.

19. The method of claim 1 wherein said wound is infected with bacteria or fungi;

20. The method of claim 19 wherein said bacteria is a Gram-negative bacteria or Gram-positive bacteria and said fungi is a filamentous fungus or yeast.

21. The method of claim 20 wherein said bacteria is a Pseudomonad or Acinetobacter, and fungus is a Candida group yeast.

22. The method of claim 21 wherein said bacteria is Pseudomonas aeruginosa, Acinetobacter baumannii, and said fungus is Candida albicans.

23. The method of claim 19 wherein said infection is reduced by at least 50%.

24. The method of claim 23 wherein said infection is reduced by at least 95%.

25. The method of claim 24 wherein said infection is reduced by at least 99.99%.

26. The method of claim 1 wherein said application to said target site occurs before said target sit is infected.
